(19) United States
(12) Patent Application Publication
(10) Pub. No.: US 2009/0123527 A1
(43) Pub. Date: May 14, 2009

(54) METHOD OF INDUCING TOPICAL ANESTHESIA AND TRANSDERMAL PATCH

(75) Inventors: ABU ALAM, Lake Forest, IL (US); Elias Reichel, Weston, MA (US); Brandon Busbee, Nashville, TN (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Akorn, Inc., Lake Forest, IL (US)

(21) Appl. No.: 12/354,422

(22) Filed: Jan. 15, 2009

Related U.S. Application Data
(63) Continuation-in-part of application No. 11/745,207, filed on May 7, 2007.
Application No. 11/745,207 is a continuation-in-part of application No. 11/491,611, filed on Jul. 24, 2006.
Application No. 11/491,611 is a continuation-in-part of application No. PCT/US07/68358, filed on May 7, 2007.

(51) Int. Cl. A61K 31/167; A61K 9/00; A61K 31/445; A61K 31/24; A61F 13/00; A61P 25/00

(52) U.S. Cl. 424/449; 514/626; 424/484; 514/330; 514/535

(57) ABSTRACT

Disclosed is a method of inducing topical anesthesia in a tissue or organ of an animal comprising providing an aqueous gel formulation comprising water, an anesthetic (e.g., lidocaine hydrochloride), a viscoelastic polymer, and a tonicity modifier, wherein the aqueous gel formulation is free of preservatives and phosphate buffer, is isotonic with physiological fluids, and is sterile and has low particulate count. Also disclosed are a transdermal patch comprising the aqueous gel formulation suitable for applying on the skin of a patient and a method of controlling pain therewith.

### CROSS-REFERENCE TO RELATED APPLICATIONS

This is a continuation-in-part of U.S. patent application Ser. No. 11/745,207 and International Patent Application No. PCT/US07/68358, both filed on May 7, 2007, which are continuation-in-part applications of U.S. patent application Ser. No. 11/491,611, filed Jul. 24, 2006. The disclosures of the '207, '358, and '611 applications are incorporated by reference.

### BACKGROUND OF THE INVENTION

Anesthesia is a process commonly used to block the perception of pain. The first public demonstration of administering an anesthetic agent occurred over 150 years ago when diethyl ether was utilized during a surgical operation to remove a tumor. Today, anesthetic agents are utilized in patient procedures across the medical specialties.

Anesthetic agents are used in procedures carried out on various tissues and organs. For example, with regard to procedures performed on the eye, common anesthetic agents utilized include subconjunctival injections of aqueous lidocaine and tetracaine drops. However, subconjunctival injections of aqueous lidocaine are less than desirable as many patients suffer from anxiety caused by needle phobia and/or the physical pain caused by the actual injection. Indeed, it is believed that the anxiety levels can reach the point where patients avoid the necessary medical care. The topical administration of tetracaine drops avoids these needle-related problems. However, there are some drawbacks with such drops. Some of the drops administered to patient may miss the eye due to the shaking of the hand or the blinking of the eye. The residence time of the drop on the eye is limited, for example, less than about a minute. Thus, the anesthetic efficacy of the tetracaine drops could become insufficient since both the onset of anesthesia is not rapid, and the duration of anesthetic activity is limited. Some of the formulations reported to be sterile do not specify the particle size limits. In addition, tetracaine may also be toxic to the cornea. Thus, there is a desire for other, more efficacious anesthetic formulations for topical administration, especially formulations which cause less anxiety, pain, and provide both rapid onset and prolonged anesthetic activity.

The invention provides such an anesthetic formulation. These and other advantages of the invention, as well as additional inventive features, will be apparent from the description of the invention provided herein.

### BRIEF SUMMARY OF THE INVENTION

The foregoing need has been fulfilled to a great extent by the invention which provides an aqueous gel formulation comprising water, an anesthetic, a viscoelastic polymer, and a tonicity modifier. Specifically, the invention provides an aqueous gel formulation which comprises, consists essentially of, or consists of, water, an anesthetic, a viscoelastic polymer, and a tonicity modifier. The formulation may also contain a pH adjusting agent or a product produced as a result of pH adjustment. Advantageously, the gel formulation is free of preservatives and/or phosphate buffer. The aqueous gel formulation of the invention is targeted for application to various tissues or organs (internal or external) of an animal, particularly to the eye of a human. The invention also provides a method for inducing topical anesthesia to a tissue or organ of an animal. The administration of the topical formulation of the invention preferably avoids the need to administer a subsequent administration (e.g., topical or injection) of the anesthetic during a medical procedure.

The aqueous gel formulation of the invention is viscous and reduces the potential for systemic absorption through nasolacrimal system, thereby reducing the risk of systemic toxicity. The aqueous gel formulation is also free of preservatives that can cause allergic reactions that are associated with corneal toxicity. The aqueous gel formulation does not cause significant corneal epithelial defects or irregularities. The aqueous gel formulation is also associated with reduced corneal drying and epithelial decompensation. With its extended corneal contact and effective anesthesia at low concentrations, the aqueous gel formulation of the invention fulfills an unmet need in the ophthalmic pharmacopeia for a topical ocular anesthetic.

The invention further provides a method of inducing topical anesthesia in a tissue or organ of an animal comprising: a) providing an aqueous gel formulation comprising water, an anesthetic, a viscoelastic polymer, and a tonicity modifier, wherein the anesthetic is present in an amount of 15 mg per ml to about 50 mg per ml of the formulation, and the gel formulation is free of preservatives and phosphate buffer, is isotonic with physiological fluids, and is sterile having less than about 100 particles of 50 microns particle size or more per ml of the aqueous gel formulation; and b) topically administering an effective amount of the aqueous gel formulation to the tissue or organ of the animal; whereby anesthesia is induced on the tissue or organ of the animal; wherein the aqueous gel formulation is administered prior to, during, or subsequent to a procedure or treatment involving bronchoscopy, colonoscopy, gastro-intestinal procedure, intubation, cannulation, dentistry, dermatology, skin procedures, skin and wound debridement, hair removal, mucosal surfaces, ear, nose, throat, urology, gynecology, herpes, oral mucositis, canker sores, prostate surgery, Botox treatment, plastic surgery, facial surgery, punch biopsy, post-operative analgesia, circumcision, inflammation, neuropathic pain, abrasions, ulcerations, lesions, incisions, trauma, joint spaces, injection sites, venipuncture, vaccination, tooth ache, teething pain, ear piercing, or body orifices.

The invention also provides a transdermal patch containing the aqueous gel formulation of the invention. The invention further provides a method of controlling localized pain comprising applying a transdermal patch to the patient's skin.

### DETAILED DESCRIPTION OF THE INVENTION

The invention provides, in an embodiment, an aqueous gel formulation comprising water, an anesthetic, a viscoelastic polymer, and a tonicity modifier, wherein the anesthetic is present in an amount of 15 mg per ml to about 50 mg per ml of the formulation, wherein the aqueous gel formulation is free of preservatives and phosphate buffer, is isotonic with physiological fluids, and is sterile having less than about 100 particles of 50 microns particle size or more per ml of the aqueous gel formulation.

The aqueous gel formulation of the invention is free of preservatives, e.g., methyl paraben, propyl paraben, or EDTA. It is also free of phosphate buffer. The aqueous gel formulation of the invention is contemplated for use on various internal and external organs of the body or tissue, particularly on the eye. In an embodiment, the gel formulation is also free of permeation enhancers such as skin permeation enhancers, e.g., glycols, surfactants, or bile salts.

In another embodiment, the invention provides an aqueous gel formulation comprising water, an anesthetic, a viscoelastic polymer, and a tonicity modifier, wherein the anesthetic is present in an amount of 25 mg per ml to about 38 mg per ml of the formulation, and is suitable for topical administration to the eye. The aqueous gel formulation is free of preservatives and phosphate buffer, is isotonic with physiological fluids, and is sterile having less than about 100 particles of 50 microns particle size or more per ml of the aqueous gel formulation.

In accordance with the invention, any suitable anesthetic can be used. Suitable anesthetics include lidocaine, bupivicaine, mepivicaine, proparacaine, and narcaine, and pharmaceutically acceptable salts thereof. Pharmaceutically acceptable salts are those derived from such organic and inorganic acids such as: acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, benzoic, and similarly known acceptable acids. Preferably, the anesthetic is lidocaine hydrochloride.

In certain embodiments, the aqueous gel formulation comprises an anesthetic in an amount of 15 mg per ml to 38 mg per ml of the formulation. Typically, the aqueous gel formulation comprises an anesthetic in an amount of 20 mg per ml to 35 mg per ml of the formulation, preferably, in an amount of 25 mg per ml to 30 mg per ml of the formulation, and more preferably, in an amount of about 35 mg per ml of the formulation.

The viscoelastic polymer comprises any suitable gelling agent. Suitable gelling agents include hydroxypropylmethylcellulose, methylcellulose, sodium carboxymethyl cellulose, ethylene oxide/propylene oxide copolymers, alginates, hyaluronates, guaran, pectin, tragacanth, carubin, carrageenan, and polyacrylic acid. Preferably, the gelling agent is hydroxypropylmethylcellulose.

The aqueous gel formulation can have any suitable pH. A suitable pH includes from about 5.0 to about 7.5, preferably, from about 5.5 to about 7.0, and more preferably, from about 6.0 to about 6.5. The pH is adjusted to minimize local, focal point irritation. The aqueous gel formulation may contain an acid or base used to adjust the pH, or any reaction product formed as a result of pH adjustment.

The aqueous gel formulation can have any suitable viscosity to enable drop-wise administration to the eye, for example, from about 2000 to about 10,000 cps, preferably from about 5000 to about 8000 cps, at 25° C. An advantage of the formulation of the invention is that, in view of the viscous nature of the formulation, the residence time of the formulation on the tissue or organ, e.g., the eye, of the patient is increased compared to anesthetic solutions which are less viscous. The increased residence time translates to long lasting anesthetic activity.

The aqueous gel formulation includes any suitable tonicity modifier to match the osmolarity (milliosmolar or mosm) of the physiological fluids. Suitable tonicity modifiers include sodium chloride, potassium chloride, mannitol, sucrose, lactose, fructose, maltose, dextrose, dextrose anhydrous, propylene glycol and glycerol. Preferably, the tonicity modifier is sodium chloride. The tonicity modifier can be present in an amount of from about 0.5 to about 1% by weight, preferably from about 0.8 to about 1% by weight, of the gel formulation. For example, the tonicity modifier, particularly sodium chloride, can be present in an amount of 0.9% by weight of the aqueous gel formulation. Typically, the aqueous gel formulation has a tonicity of from about 250 to about 350 mosm, particularly about 280 mosm. The tonicity helps to avoid hyper/hypo tonicity effects on the tissue or organ, particularly on the corneal layer, thereby increasing patient comfort.

In an embodiment, the invention provides an aqueous gel formulation consisting essentially of water, lidocaine hydrochloride, hydroxypropylmethylcellulose, and sodium chloride, wherein the lidocaine hydrochloride is present in an amount so as to provide a 1.5%, 2.5%, or 3.5% by weight of lidocaine in the formulation, wherein the aqueous gel formulation is isotonic with physiological fluids and is sterile having less than about 100 particles of 50 microns particle size or more per ml of the aqueous gel formulation. The invention also provides a method of inducing topical anesthesia comprising topically administering the aqueous gel formulation to the eye of a human.

The aqueous gel formulation can be prepared by any suitable method. For example, an aqueous solution containing the desired quantity of the viscoelastic polymer (gelling agent) and an aqueous solution containing the desired quantity of the anesthetic agent, the tonicity modifier, and pH adjusting agent, can be prepared separately. The solution containing the anesthetic and other ingredients can be sterile filtered on a 0.2 micron filter. The solution containing the viscoelastic polymer (gelling agent) is sterilized, e.g., by ethylene oxide or gamma radiation. The two solutions can be combined and mixed, and if desired diluted, to obtain an embodiment of the aqueous gel formulation of the invention.

Another aspect of the invention is a method of inducing topical anesthesia on a tissue or organ of an animal. The method comprises (a) providing an aqueous gel formulation comprising water, an anesthetic, a viscoelastic polymer, and a tonicity modifier, wherein the anesthetic is present in an amount of 15 mg per ml to about 50 mg per ml of the formulation, wherein the gel formulation is free of preservatives and phosphate buffer, is isotonic with physiological fluids, and is sterile having less than about 100 particles of 50 microns particle size or more per ml of the aqueous gel formulation; and (b) topically administering an effective amount of the aqueous gel formulation to the tissue or organ of the animal; whereby anesthesia is induced on the tissue or organ of the animal. The aqueous gel formulation can be administered in any suitable manner. For example, it can be administered drop-wise from a dropper, by a cotton-tipped applicator, or by a caulking gun or similar device.

In an embodiment, the invention provides a method of inducing topical anesthesia on the eye of an animal comprising providing an aqueous gel formulation comprising water, an anesthetic, a viscoelastic polymer, and a tonicity modifier, wherein the anesthetic is present in an amount of 25 mg per ml to 38 mg per ml of the formulation, and suitable for topical administration to the eye; and topically administering an effective amount of the aqueous gel formulation to the eye of the animal; whereby anesthesia is induced on the eye of the animal.

In any of the embodiments, upon topical administration of the aqueous gel formulation of the invention to the tissue or organ of the animal, anesthesia onsets within 5 minutes, e.g., within about 15 seconds to about 3 minutes of administration, particularly within about 5 seconds to about 1 minute of administration, or more particularly within about one second to about 30 seconds of administration. The onset time, particularly on the eye, is independent of the concentration of the anesthesia.

In a particular embodiment, where lidocaine is the anesthetic, the onset of anesthesia takes place within a period of about 20 seconds or more, 40 seconds or more, 1 minute or more, or 2 minutes or more. The mean time to anesthetic onset for a formulation of the invention containing lidocaine ranges from about 40 seconds to about 60 seconds in the concentration range of from 1.5% to 3.5% by weight.

Anesthesia induced on the tissue or organ after administration of the aqueous gel formulation lasts up to 30 minutes or more, e.g., up to about 10 to 30 minutes, up to about 15 to 20 minutes, or up to about 25 minutes, so as to permit completion of a lengthy procedure, for example, cataract surgery. The duration of activity is dependent upon the concentration of the anesthetic. For example, the duration is 30 minutes or more at 3.5%; 20 minutes or more at 2.5%, and 10 minutes or more at 1.5% concentration of the anesthetic by weight. The embodiments of the invention possess advantageous properties including rapid onset of topical anesthesia and prolonged anesthetic activity, enabling various medical and surgical procedures to proceed without undesirable intervention, e.g., an anesthetic injection.

For example, the 1.5% formulation can be used in situations that require topical anesthesia for approximately 5 to 10 minutes. This duration of anesthesia is advantageous for office based ophthalmic procedures and tests. The 1.5% formulation can be used with diagnostic contact lenses such as those used for examination of the peripheral retina, gonioscopy, and electroretinographic testing. When lenses are used in these procedures, typically both anesthetic and methylcellulose gels are used to provide patient comfort and provide a view for the ophthalmologist by contact biomicroscopy. An embodiment of the present invention, e.g., the 1.5% formulation can accomplish both anesthesia and serve as an optical coupling agent with one application. Most office based laser procedures and diagnostic tests are typically performed in less than 5 minutes. Other minor surgical procedures that typically last less than 10 minutes include intravitreal injections for macular degeneration and diabetic patients, subtenon injections, conjunctival biopsies, pterygium removal with autografting of conjunctiva, suture removal, removal of corneal and conjunctival foreign bodies, incision and drainage of chalazia, and lid injections.

For procedures that require longer duration, a formulation having higher anesthesia contents can be used, for example, a formulation containing 3.5% anesthetic, e.g., lidocaine. Intraocular surgeries, such as cataract, trabeculectomies, and pars plana vitrectomies are increasingly being dome using topical anesthesia, either as primary or adjunctive anesthesia, with sedation. Refractive surgery and suture adjustments after strabismus surgery are also performed under topical anesthesia. These procedures typically require a longer anesthesia time than that is required for many office based procedures. The 3.5% formulation can provide longer anesthesia, e.g., 10-30 minutes, while multiple applications every 10-15 minutes can allow for more extended procedures.

Embodiments of the aqueous gel formulation of the invention possess long term storage stability, for example, they are stable for a period of up to 1, 2, 3 months or more, e.g., 24 months or more, at 40° C. and 20% relative humidity (RH). The aqueous gel formulation of the invention possesses freeze/thaw stability. The aqueous gel formulation of the invention advantageously has long term stability such that the assay of the anesthetic is within 95.0% to 105.0%; not more than 0.1% large anesthetic degradents (particularly large lidocaine degradent); and not more than 1.0% of total anesthetic degradents (particularly total lidocaine degradent) over 3 months at 40° C. and 20% RH. The degradents can be measured by any suitable method, e.g., HPLC. In embodiments of the invention, particularly where lidocaine hydrochloride is used on the eye, the aqueous gel formulation is clear, colorless, and free or substantially free from undissolved material or particulates.

Embodiments of the aqueous gel formulation of the invention have the advantage of decreased risk of post-surgical endophthalmitis and/or decreased corneal toxicity. It is contemplated that the formulation of the invention provides a superior anesthetic property over 0.5% tetracaine, and does not require a subconjunctival injection prior to treatment with intravitreal injection. In addition, lidocaine is less toxic to the cornea than tetracaine.

As the aqueous gel formulation of the invention is free of preservatives and is targeted for single use, it provides for increased patient safety. There is a decreased probability of cross contamination and irritation on the tissue or organ, particularly on the corneal/epithelial layer of the eye. Preservatives, such as parabens, tend to degrade, e.g., hydrolyze to the corresponding acid (p-hydroxybenzoic acid) and alcohols (e.g., methanol, ethanol, or propanol). Since the formulation is free of preservatives, the possibility of degradents being present in the formulation is decreased, and therefore, any adverse effect due to such degradents is decreased. Advantageously, the time to onset of anesthetic activity is independent of concentration of the anesthetic. The duration of anesthetic activity can be controlled by controlling the concentration of the anesthetic. Advantageously, the aqueous gel formulation of the invention contains the anesthetic and the viscoelastic polymer in a dissolved molecular state, thereby permitting constant rate of release of the anesthetic over time. This leads to increased duration of anesthetic activity and patient comfort. In addition, controlling the particle size of impurities and their number as well as reducing degradents to a minimum increases corneal safety.

The aqueous gel formulation of the invention is contemplated for use on procedures carried out on various tissues and organs, e.g., in bronchoscopy, colonoscopy, GI procedures, intubation, dentistry, dermatology, skin procedures, mucosal surfaces, ear, nose, and throat (ENT), urology, and gynecology.

In accordance with an embodiment, the invention provides a method of inducing topical anesthesia in a tissue or organ of an animal comprising:

a) providing an aqueous gel formulation comprising water, an anesthetic, a viscoelastic polymer, and a tonicity modifier, wherein the anesthetic is present in an amount of 15 mg per ml to about 50 mg per ml of the formulation, and the gel formulation is free of preservatives and phosphate buffer, is isotonic with physiological fluids, and is sterile having less than about 100 particles of 50 microns particle size or more per ml of the aqueous gel formulation; and

b) topically administering an effective amount of the aqueous gel formulation to the tissue or organ of the animal;

whereby anesthesia is induced on the tissue or organ of the animal,

wherein the aqueous gel formulation is administered prior to, during, or subsequent to a procedure or treatment involving bronchoscopy, colonoscopy, gastro-intestinal procedure, intubation, cannulation, dentistry, dermatology, skin procedures, skin and wound debridement, hair removal, mucosal surfaces, ear, nose, throat, urology, gynecology, herpes, oral mucositis, canker sores, prostate surgery, Botox treatment, plastic surgery, facial surgery, punch biopsy, post-operative analgesia, circumcision, inflammation, neuropathic pain, abrasions, ulcerations, lesions, incisions, trauma, joint spaces, injection sites (e.g., pediatric injection sites), venipuncture, vaccination, tooth ache, teething pain, ear piercing, or body orifices. For hair removal, the aqueous gel formulation can be placed on an adhesive sheet.

The aqueous gel formulation of the invention can be filled for single use in any suitable size container, for example, 5 ml dropper bottles, using aseptic techniques.

In an embodiment, the invention provides a method of inducing topical anesthesia on the eye of an animal, e.g., human. Thus, it is contemplated that the aqueous gel formulation can be administered to the eye prior to or during a variety of procedures performed on the eye, for example, a procedure selected from the group consisting of intravitreal injection, conjunctival or corneal foreign body removal, gonioscopy, suture placement, removal of corneal sutures, removal of conjunctival sutures, removal of lid sutures, anterior chamber paracentesis, contact lens examination of retina, a procedure involving ALT/SLT laser, a procedure involving retinal lasers, placement of electroretinographic lenses, lens placement for YAG laser, scleral depression examination, cataract surgery, refractive surgery, supplemental topical anesthetic after peribulbar or retrobulbar block, vitreous biopsy, conjunctival biopsy, minor lid procedure, retinal cryoretinopexy, pneumatic retinopexy, pterygium surgery, strabismus surgery adjustment, conductive keratoplasty, pars plana vitrectomy, and trabeculectomy, and any combination thereof. If needed, additional anesthetic formulation can be administered.

In accordance with an embodiment of the invention, a formulation containing 15 mg/mL anesthesia, e.g. lidocaine, can be administered to the eye prior to or during a procedure selected from the group consisting of intravitreal injection, conjunctival or corneal foreign body removal, gonioscopy, suture placement, removal of corneal sutures, removal of conjunctival sutures, removal of lid sutures, anterior chamber paracentesis, contact lens examination of retina, a procedure involving ALT/SLT laser, a procedure involving retinal lasers, placement of electroretinographic lenses, lens placement for YAG laser, and scleral depression examination, and any combination thereof.

In accordance with another embodiment of the invention, an aqueous gel formulation having containing 35 mg/mL of anesthetic, e.g., lidocaine, can be administered to the eye prior to or during a procedure selected from the group consisting of cataract surgery, refractive surgery, supplemental topical anesthetic after peribulbar or retrobulbar block, vitreous biopsy, conjunctival biopsy, minor lid procedure, retinal cryoretinopexy, pneumatic retinopexy, pterygium surgery, strabismus surgery adjustment, conductive keratoplasty, pars plana vitrectomy, and trabeculectomy, and any combination thereof.

The invention further provides a transdermal patch containing an aqueous gel formulation in accordance with any of the embodiments described above. For example, the invention provides a transdermal patch containing an aqueous gel formulation comprising water, an anesthetic, a viscoelastic polymer, and a tonicity modifier, wherein the anesthetic is present in an amount of 15 mg per ml to about 50 mg per ml of the formulation, and the gel formulation is free of preservatives and phosphate buffer, is isotonic with physiological fluids, and is sterile having less than about 100 particles of 50 microns particle size or more per ml of the aqueous gel formulation.

The transdermal patch includes a backing layer, an adhesive, a release liner, and a membrane, wherein the membrane and the backing layer forming a drug reservoir containing the aqueous gel formulation. In accordance with an embodiment, the adhesive is disposed on the backing layer. The release liner protects the patch during storage and is removed prior to use.

The transdermal patch or skin patch can be used to control localized pain, e.g., superficial pain or neuronal pain, such as lower back pain including post-herpetic neuralgia, and arthritis pain.

A permeable membrane, which allows the gel formulation to be delivered at a controlled rate to the patient, separates the aqueous gel formulation in the drug reservoir and the release liner. Examples of membranes suitable for use include ethylcellulose, polyvinyl alcohol, polyethylene, ethylene-vinyl acetate copolymer, chitosan, and cellulose acetate membranes.

In an embodiment, the adhesive is applied to the backing layer around the periphery of the drug reservoir. Adhesives such as rubbers, e.g., polyisobutylenes, vinyl acetate polymers, or acrylates, can be used to form the adhesive layer. The adhesive layer may additionally contain plasticizers such as inert, organic, apolar, nonvolatile hydrophobic liquids, e.g., long-chain aliphatic esters and alcohols, mineral oil, linseed oil, octyl palmitate, squalene, squalane, silicone oil, isobutyl stearate, olive oil, isopropyl myristate, isostearyl alcohol, oleyl alcohol, and the like. The adhesive may further include fillers such as metal oxides, e.g., zinc oxide, titanium oxide, and magnesium oxide, inorganic salts, e.g., calcium, magnesium, and sodium carbonates, synthetic polymers, clays, and the like.

The drug reservoir may contain any suitable amount of the aqueous gel formulation, for example, about 0.1, 0.2, 0.3, 0.4, 0.5 ml or more, for example, from about 0.1 ml to about 5 ml, in certain embodiments from about 0.2 ml to about 2 ml, and in certain other embodiments from about 0.3 ml to about 1 ml.

The backing layer is typically made of a polyester film, ethylene vinyl alcohol copolymer film, or a polyurethane film. The release liner is typically a polyester fabric.

The following examples further illustrate the invention but, of course, should not be construed as in any way limiting its scope.

### EXAMPLE 1

This example illustrates a method of preparing an aqueous gel formulation comprising lidocaine hydrochloride in an amount of 15 mg per ml of the formulation in accordance with an embodiment of the invention.

500 ml of purified water is charged into a sterile vessel #1 using an aseptic technique. 25 g of sterile hydroxypropylmethylcellulose is charged into vessel #1 using an aseptic technique and mixed. In a separate vessel #2, 15 g of lidocaine hydrochloride and 9 g of sodium chloride are dissolved in about 400 ml of purified water and passed through a 0.2 micron filter and aseptically transferred to vessel #1 with mixing. Hydrochloric acid and/or sodium hydroxide solutions are filtered through a 0.2 micron filter and added to vessel #1 to adjust the pH to 6.0-6.5. Purified water is passed through a 0.2 micron filter to bring the formulation to 1 kg. The formulation is a sterile viscous gel and may be filled into sterile unit dose bottles of suitable size, e.g., 5 ml dropper bottles, using aseptic technique.

### EXAMPLE 2

This example illustrates a method of preparing an aqueous gel formulation comprising lidocaine hydrochloride in an amount of 25 mg per ml of the formulation in accordance with an embodiment of the invention.

500 ml of purified water is charged into a sterile vessel #1 using an aseptic technique. 25 g of sterile hydroxypropylmethylcellulose is charged into vessel #1 using an aseptic technique and mixed. In a separate vessel #2, 25 g of lidocaine hydrochloride and 9 g of sodium chloride are dissolved in about 400 ml of purified water and passed through a 0.2 micron filter and aseptically transferred to vessel #1 with mixing. Hydrochloric acid and sodium hydroxide solutions are filtered through a 0.2 micron filter and added to vessel #1 to adjust pH 6.0-6.5. Purified water is passed through a 0.2 micron filter to bring the formulation to 1 kg. The formulation is a sterile viscous gel and may be filled into sterile unit dose bottles of suitable size, e.g., 5 ml dropper bottles, using aseptic technique.

### EXAMPLE 3

This example illustrates a method of preparing an aqueous gel formulation comprising lidocaine hydrochloride in an amount of 35 mg per ml of the formulation in accordance with an embodiment of the invention.

500 ml of purified water is charged into a sterile vessel #1 using aseptic technique. 25 g of sterile hydroxypropylmethylcellulose is charged into vessel #1 using aseptic technique and mixed. In a separate vessel #2, 35 g of lidocaine hydrochloride and 9 g of sodium chloride are dissolved in about 400 ml of purified water and passed through a 0.2 micron filter and aseptically transferred to vessel #1 with mixing. Hydrochloric acid and sodium hydroxide solutions are filtered through a 0.2 micron filter and added to vessel #1 to adjust pH 6.0-6.5. Purified water is passed through a 0.2 micron filter to bring the formulation to 1 kg. The formulation is a sterile viscous gel and may be filled into sterile unit dose bottles of suitable size, e.g., 5 ml dropper bottles, using aseptic technique.

### EXAMPLE 4

This example illustrates the long term stability and freeze/thaw stability of an embodiment of the aqueous gel formulation of the invention comprising lidocaine hydrochloride in an amount of 15 mg per ml of the formulation.

An aqueous gel formulation comprising lidocaine hydrochloride in an amount of 15 mg per ml of the formulation is placed on accelerated stability storage at 40° C. and 20% relative humidity. The lidocaine hydrochloride formulation and potential degradents are measured initially and after 1, 2, and 3 months of accelerated storage using a high pressure liquid chromatographic method. There is no change of the formulation characteristics, assay values, and degradents upon accelerated storage supporting a room temperature stability of 24 months. The freeze thaw cycle data also show the formulation to be stable during transportation and extreme seasonal exposures to temperatures. The stability data is shown in Table 1.

TABLE 1
Stability data for aqueous gel formulation

| Test | Limits | Initial | 1 mo. | 2 mo. | 3 mo. | Freeze/thaw |
|---|---|---|---|---|---|---|
| Assay: | 95.0%-105.0% | 99.7% | 100.4% | 99.6% | 98.0% | 97.1% |
| Lidocaine | | | | | | |
| Hydrochloride | | | | | | |
| Large | NMT: 0.1% | 0.06% | 0.07% | 0.06% | 0.07% | 0.08% |
| Lidocaine | | | | | | |
| Degradent | | | | | | |
| Total | NMT: 1.0% | 0.06% | 0.07% | 0.06% | 0.07% | 0.08% |
| Lidocaine | | | | | | |
| Degradent | | | | | | |
| Minimum Fill | NLT: 5 ml | 6.3 ml | 6.2 ml | 6.2 ml | 6.2 ml | 6.3 ml |
| pH | 5.5-7.5 | 6.3 | 6.0 | 5.9 | 5.8 | 6.0 |
| Appearance | Clear, | Pass | Pass | Pass | Pass | Pass |
| | colorless | | | | | |
| | solution; | | | | | |
| | free from | | | | | |
| | undissolved | | | | | |
| | material | | | | | |
| Sterility | Sterile | Pass | N/A | N/A | N/A | N/A |

### EXAMPLE 5

This example illustrates the long term stability and freeze/thaw stability of an embodiment of the aqueous gel formulation of the invention comprising lidocaine hydrochloride in an amount of 25 mg per ml of the formulation.

An aqueous gel formulation comprising lidocaine hydrochloride in an amount of 25 mg per ml of the formulation is placed on accelerated stability storage at 40° C. and 20% relative humidity. The lidocaine hydrochloride formulation and potential degradents are measured initially and after 1, 2, and 3 months accelerated storage using a high pressure liquid chromatographic method. There is no change of the formulation characteristics, assay values and degradents upon accelerated storage supporting a room temperature stability of 24 months. The freeze thaw cycle data also show the formulation to be stable during transportation and extreme seasonal exposures to temperature. The stability data is shown in Table 2.

TABLE 2
Stability data for aqueous gel formulation

| Test | Limits | Initial | 1 mo. | 2 mo. | 3 mo. | Freeze/thaw |
|---|---|---|---|---|---|---|
| Assay: | 95.0%-105.0% | 99.3% | 100.6% | 101.1% | 98.7% | 98.3% |
| Lidocaine | | | | | | |
| Hydrochloride | | | | | | |
| Large | NMT: 0.1% | 0.06% | 0.07% | 0.08% | 0.06% | 0.07% |
| Lidocaine | | | | | | |
| Degradent | | | | | | |
| Total | NMT: 1.0% | 0.07% | 0.07% | 0.08% | 0.06% | 0.14% |
| Lidocaine | | | | | | |
| Degradent | | | | | | |
| Minimum Fill | NLT: 5 ml | 6.5 ml | 6.4 ml | 6.4 ml | 6.4 ml | 6.5 |
| pH | 5.5-7.5 | 6.4 | 6.0 | 6.0 | 5.9 | 6.1 |
| Appearance | Clear, | Pass | Pass | Pass | Pass | Pass |
| | colorless | | | | | |
| | solution; | | | | | |
| | free from | | | | | |
| | undissolved | | | | | |
| | material | | | | | |
| Sterility | Sterile | Pass | N/A | N/A | N/A | N/A |

### EXAMPLE 6

This example illustrates the long term stability of an embodiment aqueous gel formulation of the invention comprising lidocaine hydrochloride in an amount of 35 mg per ml of the formulation.

An aqueous gel formulation comprising lidocaine hydrochloride in an amount of 35 mg per ml of the formulation is placed on accelerated stability storage at 40° C. and 20% relative humidity. The lidocaine hydrochloride and potential degradents are measured initially and after 1, 2, and 3 months accelerated storage using a high pressure liquid chromatographic method. There is no change of the formulation characteristics, assay values and degradents upon accelerated storage supporting a room temperature stability of 24 months. The freeze thaw cycle data also show the formulation to be stable during transportation and extreme seasonal exposures to temperature. The stability data is shown in Table 3.

TABLE 3
Stability data for aqueous gel formulation

| Test | Limits | Initial | 1 mo. | 2 mo. | 3 mo. | Freeze/thaw |
|---|---|---|---|---|---|---|
| Assay: | 95.0%-105.0% | 98.9% | 100.1% | 100.4% | 98.9% | 98.5% |
| Lidocaine | | | | | | |
| Hydrochloride | | | | | | |
| Large | NMT: 0.1% | 0.05% | 0.07% | 0.07% | 0.06% | 0.05% |
| Lidocaine | | | | | | |
| Degradent | | | | | | |
| Total | NMT: 1.0% | 0.05% | 0.07% | 0.07% | 0.06% | 0.09% |
| Lidocaine | | | | | | |
| Degradent | | | | | | |
| Minimum Fill | NLT: 5 ml | 6.4 ml | 6.4 ml | 6.3 ml | 6.3 ml | 6.4 |
| pH | 5.5-7.5 | 6.3 | 6.0 | 6.0 | 5.9 | 6.1 |
| Appearance | Clear, | Pass | Pass | Pass | Pass | Pass |
| | colorless | | | | | |
| | solution; | | | | | |
| | free from | | | | | |
| | undissolved | | | | | |
| | material | | | | | |
| Sterility | Sterile | Pass | N/A | N/A | N/A | N/A |

### EXAMPLE 7

This example illustrates that aqueous gel formulations in accordance with embodiments of the invention achieve anesthetic effect on the human eye and it is safe to administer the formulations.

This is a two day, multi-centered, randomized, prospective, sham controlled study conducted at 7 study sites to assess the effectiveness of topical formulations containing lidocaine hydrochloride at 1.5% (15 mg/mL), 2.5% (25 mg/mL), and 3.5% (35 mg/mL) of lidocaine to achieve ocular surface anesthesia.

Participants are randomized 1:1:1:1 to sham, 1.5%, 2.5%, or 3.5% formulations. Following baseline fluorescein corneal staining, study participants are given 2 drops of the formulation approximately 5 mm posterior to the limbus at the 6 o'clock position. Simultaneously, a timer is started. At the 20-second mark, the conjunctiva is tested with a 0.3 forceps at the center of the applied formulation. The study subject is instructed to state ‘pain’ if there is any pain with pinching of the conjunctiva with the forceps. If there is no pain or only pressure, the subject does not respond. This technique is to be repeated at 20-second intervals until anesthesia is achieved (i.e., no response from the study subject). Subjects who indicate they have no pain (indicating anesthesia) are then tested at 5-minute intervals starting at the 5-minute mark. The testing is concluded when the study subject reports ‘pain’ on two successive tests.

If the study subject reports ‘pain’ at both the 20 second mark and 40 second mark, testing is performed at the 1 minute mark. If the subject reports ‘pain’ at 1 minute, testing is suspended until be 5 minute mark. If the subject reports ‘pain’ at the 5 minute mark, no more conjunctival pinching is performed and it is deemed that anesthesia is not achieved. Subjects return to the clinical site on the day following treatment (Day 2) for follow-up examinations.

Two-hundred subjects are planned (50 per treatment group). A total of 209 subjects are enrolled, with 54, 51, 53, and 51 subjects randomized to the sham and the formulation (1.5%, 2.5%, and 3.5%) treatment groups, respectively. All subjects complete the study and are analyzed or safety and efficacy. Subjects 18 years of age or older who have a condition that requires ocular anesthesia are eligible for this study. Subjects who have undergone ocular surgery within 4 weeks of the study and those with evidence of ocular inflammation or other ocular conditions that could interfere with the study assessments are excluded. Each subject receives 2 drops of the study article (sham or test formulation) on Day 1 of the study.

The primary efficacy variable, i.e., percentage of subjects who achieve ocular surface anesthesia within 5 minutes post-application of the formulation, and the secondary efficacy variables, which include the time of onset and the duration of ocular surface anesthesia, are determined. Subject safety is assessed through the monitoring and reporting of any adverse events (AEs) that occur during the study. The frequency and severity of the AE profiles for each treatment group are evaluated to show their comparability. Slit lamp eye examinations are conducted to assess for clinically significant treatment-emergent changes.

A two-sided hypothesis testing is conducted for tests. The resulting p-values less than or equal to 0.05 are considered statistically significant. SAS software is used for the data analyses and tabulations. The pain evaluation determines the time to first “No Pain” response and the time at which two successive corneal pinches result in a “Pain” response. If a “No Pain” response is not reported by 5 minutes post-treatment, no further pain evaluation is made. The primary efficacy variable is coded as follows: 1 for “No Pain” prior to 5 minutes after application, and 2 for “Pain” observed at all study time points up to and including 5 minutes. Statistical analysis is conducted using intent-to-treat (ITT) and per-protocol (PP) subject populations. The primary efficacy variable is analyzed using the normal approximation to the odds ratio of each level of treatment with sham. The significance associated with the test H:odds ratio=1 is rejected when the p-value exceeds the critical value for Dunnett's test for alpha=0.05 and four comparisons are made with sham.

The secondary efficacy variables (time of onset and duration of ocular surface anesthesia) are analyzed using the analysis of covariance, in which each variable is regressed on treatment and combined center. Dunnett's test is used to assess the significance of the resulting comparisons of treatment with sham. For duration of anesthesia, the time difference between the time anesthesia is first achieved and the first report of pain is used. If anesthesia is not achieved, duration of anesthesia is imputed to zero. As the duration of anesthesia results are not normally distributed, an additional non-parametric analysis is conducted to explore the relationship between dose and duration of anesthesia. The incidence of AEs is summarized by treatment group and compared using descriptive statistics. No hypothesis testing is performed for the safety variables. The results are set forth below:

TABLE 4
Primary and Secondary Efficacy Analyses of
the Effect of the Aqueous Gel Formulations

| | | | | |
|---|---|---|---|---|
| | 1.5% | 2.5% | 3.5% | |
| | formu- | formu- | formu- | |
| Sham | lation | lation | lation | Overall |
| (N = 54) | (N = 51) | (N = 53) | (N = 51) | (N = 209) |

Percent Achieving Anesthesia within 5 Minutes of Dosing

| | | | | | |
|---|---|---|---|---|---|
| N (%) | 12 (22) | 45 (88) | 47 (89) | 47 (92) | 151 (72) |
| p-values1 | | <0.001 | <0.001 | <0.001 | <0.001 |

Duration of

| | | | | | |
|---|---|---|---|---|---|
| Mean | 171.2 | 614.3 | 823.1 | 801.8 | 598.5 |
| S.D. | 433.48 | 458.54 | 1074.76 | 497.46 | 719.12 |
| Median | 0.0 | 561.0 | 580.0 | 620.0 | 560.0 |
| Min | 0 | 0 | 0 | 0 | 0 |
| Max | 2062 | 2360 | 7192 | 2080 | 7192 |
| p-values | | 0.001 | <0.001 | <0.001 | <0.001 |

Time to Anesthesia

| | | | | | |
|---|---|---|---|---|---|
| Mean | 85.0 | 46.6 | 59.8 | 58.2 | 57.4 |
| S.D. | 101.67 | 57.18′ | 89.34 | 75.99 | 77.67 |
| Median | 50.0 | 40.0 | 20.0 | 40.0 | 40.0 |
| Min | 20 | 15 | 20 | 20 | 15 |
| Max | 300 | 301 | 360 | 302 | 360 |

P-values below treatment are Dunnett's adjusted comparison with Sham. P-value below overall is the test of homogeneity of all treatments. S.D. = standard deviation; Min = minimum; Max = maximum
NOTE:
Duration and Time to Anesthesia are in seconds.

TABLE 5
Cumulative Frequency (%) of Subjects Achieving
Anesthesia by Onset Time and Treatment

| | Sham | | 1.5% formulation | | 2.5% formulation | | 3.5% formulation | |
|---|---|---|---|---|---|---|---|---|
| Onset Time | (N = 54) | | (N = 51) | | (N = 53) | | (N = 51) | |
| (sec) | N (%) of Subjects | | | | | | | |
| 20 | 3 | (25%) | 16 | (35.56%) | 25 | (53.19%) | 16 | (34.04%) |
| 40 | 6 | (50%) | 34 | (75.56%) | 37 | (78.72%) | 35 | (74.47%) |
| 60 | 10 | (83.33%) | 43 | (95.56%) | 41 | (87.23%) | 41 | (87.23%) |
| 300 [1] | 12 | (100%) | 45 | (100%) | 46 | (97.87%) [2] | 47 | (100%) |

| | | | | |
|---|---|---|---|---|
| Anesthesia | 42 | 6 | 6 | 4 |

not achieved
[1] 300 second time point also includes assessments conducted up to 304 seconds.
[2] Excludes one subject who achieved anesthesia at 360 seconds
% cumulative frequency based on the number of subjects achieving anesthesia within 5 minutes

TABLE 6
Summary of Duration of Anesthesia Excluding Outlier Value

| | Sham | Akten ® 1.5% | Akten 2.5% | Akten 3.5% |
|---|---|---|---|---|
| Time (sec) | N = 54 | N = 51 | N = 52 | N = 51 |
| Mean | 171.2 | 614.3 | 700.6 | 801.8 |
| S.D. | 433.48 | 458.54 | 605.90 | 497.46 |
| Median | 0.0 | 561.0 | 580.0 | 620.0 |
| Min | 0 | 0 | 0 | 0 |
| Max | 2062 | 2360 | 3280 | 2080 |
| P-values ¹ | | <0.0001 | <0.0001 | <0.0001 |

¹ P-values below treatment are Dunnett's adjusted comparison with Sham Subject 06/0026 in the Akten 2.5% group excluded from summary statistics

The proportion of subjects who achieve anesthesia in 5 minutes is comparable across the groups that are administered the formulation. Anesthesia is achieved by 45 of 51 subjects (88%), 47 of 53 subjects (89%), and 47 of 51 subjects (92%), respectively, in the 1.5%, 2.5%, and 3.5% formulation treatment groups. Only 12 of the 54 subjects (22%) in the sham group achieve anesthesia. The mean time to anesthesia onset is not affected by dose. Anesthesia onset times range from 20 seconds to 5 minutes, and the mean time to anesthesia onset is 85 seconds, 46.6 seconds, 59.8 seconds, and 58.2 seconds, respectively, for the sham and 1.5%, 2.5%, and 3.5% formulation treatment groups. Among the subjects who achieve anesthesia within 5 minutes, 83% 96%, 87%, and 87% in the sham and 1.5%, 2.5%, and 3.5% formulation treatment groups respectively, achieve anesthesia within 60 seconds of application.

Across all treatment groups, duration of anesthesia range from 0 to 7192 seconds. Mean durations for the 1.5%, 2.5%, and 3.5% formulation treatment groups (614 seconds, 823 seconds, and 802 seconds, respectively) are significantly longer (p<0.001) than those of the sham group (171 seconds). When the outlier value of 7192 seconds in the 2.5% formulation treatment group is excluded, duration of anesthesia demonstrates a clear pattern of increasing anesthesia duration with increasing dose. Among subjects who achieve anesthesia, mean anesthesia durations are 696 seconds (approximately 12 minutes), 792 seconds (approximately 13 minutes), and 870 seconds (approximately 15 minutes) for the 1.5%, 2.5%, and 3.5% formulation treatment groups, respectively. Approximately 70%, 75%, and 85% of the subjects in the 1.5% 2.5%, and 3.5% formulation treatment groups, respectively, experience anesthesia for at least 5 minutes, and approximately 35%, 42%, and 55% of the subjects experience anesthesia for 10 minutes or longer and 16%, 23%, and 27% of the subjects experience anesthesia for 15 minutes or longer. Doses of the 1.5%, 2.5%, and 3.5% formulations are well tolerated by the subjects in this study, and the incidence of AEs is low and comparable across dose groups. Across all treatment groups, the most frequently occurring AEs are conjunctival hyperemia (13 subjects [6%]) and conjunctival hemorrhage (7 subjects [3%]). Conjunctival hyperemia is reported by four subjects each (8%) in the 1.5%, 2.5%, and 3.5% formulation treatment groups and by 1 subject (2%) in the sham group. Conjunctival hemorrhage is reported by 3 subjects (6%), 1 subject (2%), and 3 subjects (6%) in the 1.5%, 2.5%, and 3.5% formulation treatment groups, respectively, and is most likely related to pinching of the conjunctiva with forceps to determine whether anesthesia has been achieved. Corneal staining is reported by 3 subjects (6%) in the 3.5% formulation treatment group and 1 subject (2%) in the sham group, and headache is reported by 1 subject each (2%) in the 1.5%, 2.5%, and 3.5% formulation treatment groups. All other AEs (eye pain, lacrimal disorder, and hyperhidrosis) are reported by 1 subject (<1.0%) each. All but 3 AEs of headache and one AE of hyperhidrosis are related to eye disorders and are consistent with the study article and pain assessment procedures. The majority of AEs is mild or moderate and resolves without treatment within 24 hours. No evidence of allergic reaction or corneal laziness is observed. With the exception of 4 AEs of corneal staining, the results of slit lamp eye examinations do not reveal any clinically significant changes following treatment.

The 1.5%, 2.5%, and 3.5% formulations are well tolerated and no dose-related corneal toxicity is observed. Approximately 90% of subjects achieve ocular anesthesia within 5 minutes following application of 1.5%, 2.5%, and 3.5% formulations, with 90% of these subjects experiencing anesthesia within 60 seconds. The duration of anesthesia increases with increasing dose, suggesting the potential for two distinct anesthetic durations of this formulation, thereby allowing physicians to tailor the anesthetic needs of the patient to the clinical situation.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of inducing topical anesthesia in a tissue or organ of an animal comprising:
a) providing an aqueous gel formulation comprising water, an anesthetic, a viscoelastic polymer, and a tonicity modifier, wherein the anesthetic is present in an amount of 15 mg per ml to about 50 mg per ml of the formulation, and the gel formulation is free of preservatives and phosphate buffer, is isotonic with physiological fluids, and is sterile having less than about 100 particles of 50 microns particle size or more per ml of the aqueous gel formulation; and
b) topically administering an effective amount of the aqueous gel formulation to the tissue or organ of the animal;
whereby anesthesia is induced on the tissue or organ of the animal,
wherein the aqueous gel formulation is administered prior to, during, or subsequent to a procedure or treatment involving bronchoscopy, colonoscopy, gastro-intestinal procedure, intubation, cannulation, dentistry, dermatology, skin procedures, skin and wound debridement, hair removal, mucosal surfaces, ear, nose, throat, urology, gynecology, herpes, oral mucositis, canker sores, prostate surgery, Botox treatment, plastic surgery, facial surgery, punch biopsy, post-operative analgesia, circumcision, inflammation, neuropathic pain, abrasions, ulcerations, lesions, incisions, trauma, joint spaces, injection sites, venipuncture, vaccination, tooth ache, teething pain, ear piercing, or body orifices.

2. The method of claim 1, wherein the anesthetic is selected from the group consisting of lidocaine, bupivicaine, mepivicaine, proparacaine, and narcaine, and salts thereof.

3. The method of claim 2, wherein the anesthetic is lidocaine hydrochloride.

4. The method of claim 1, wherein the anesthetic is present in an amount of from 15 mg to 38 mg per ml and the aqueous gel formulation is suitable for administration to the eye.

5. The method of claim 1, wherein the viscoelastic polymer comprises a gelling agent.

6. The method of claim 5, wherein the gelling agent is hydroxypropylmethylcellulose.

7. The method of claim 1, wherein the aqueous gel formulation has a pH from about 5.0 to about 7.5.

8. The method of claim 1, wherein the aqueous gel formulation has a viscosity from about 2000 to about 10,000 cps at 25° C.

9. The method of claim 1, wherein the tonicity modifier is sodium chloride.

10.-11. (canceled)

12. The method of claim 3, wherein the lidocaine hydrochloride is present in an amount so as to provide a 1.5%, 2.5%, or 3.5% by weight of lidocaine in the aqueous gel formulation.

13. (canceled)

14. A transdermal patch containing an aqueous gel formulation comprising water, an anesthetic, a viscoelastic polymer, and a tonicity modifier, wherein the anesthetic is present in an amount of 15 mg per ml to about 50 mg per ml of the formulation, and the gel formulation is free of preservatives and phosphate buffer, is isotonic with physiological fluids, and is sterile having less than about 100 particles of 50 microns particle size or more per ml of the aqueous gel formulation.

15. The transdermal patch of claim 14, which includes a backing layer, an adhesive, a release liner, and a membrane, wherein said membrane and said backing layer forming a drug reservoir containing the aqueous gel formulation.

16. The transdermal patch of claim 15, wherein the adhesive is disposed on the backing layer.

17. The transdermal patch of claim 14, wherein the anesthetic is selected from the group consisting of lidocaine, bupivicaine, mepivicaine, proparacaine, and narcaine, and salts thereof.

18. The transdermal patch of claim 14, wherein the anesthetic is lidocaine hydrochloride.

19. The transdermal patch of claim 14, wherein the anesthetic is present in an amount of from 15 mg to 38 mg per ml of the aqueous gel formulation.

20.-22. (canceled)

23. The transdermal patch of claim 14, wherein the aqueous formulation has a viscosity from about 2000 to about 10,000 cps at 25° C.

24. (canceled)

25. The transdermal patch of claim 18, wherein the lidocaine hydrochloride is present in an amount so as to provide a 1.5%, 2.5%, or 3.5% by weight of lidocaine in the aqueous gel formulation.

26. A method of controlling localized pain of a patient comprising applying to skin of the patient a transdermal patch of claim 14.

27. The method of claim 26, wherein the pain is due to arthritis, lower back or post-herpetic neuralgia.
